(19) United States
(12) Patent Application Publication
(10) Pub. No.: US 2008/0299671 A1
(43) Pub. Date: Dec. 4, 2008

(54) Hydrophobic Interaction Chromatography

(75) Inventors: Gunnar Glad, Uppsala (SE); Bo-Lennart Johansson, Uppsala (SE); Jean-Luc Maloisel, Uppsala (SE); Nils Norrman, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 12/095,752

(22) PCT Filed: Nov. 30, 2006
(86) PCT No.: PCT/SE2006/001369

§ 371 Date: Jun. 2, 2008

(30) Foreign Application Priority Data
Dec. 2, 2005 (SE) 0502683-6

(51) Int. Cl. G01N 30/02; B01D 15/16; B01D 15/32; C07K 16/06; C07K 1/00

(52) U.S. Cl. 436/161; 210/635; 530/413

(57) ABSTRACT

The present invention relates to a method of isolating target compounds from a liquid, which method including the steps of providing a mobile phase, which contains at least one target compound and wherein the conductivity corresponds to ≧0.6 M; contacting the mobile phase with a separation matrix including one or more sulphonamide groups to adsorb one or more target compounds; contacting an eluent with the matrix to release target compound(s), wherein the conductivity of the eluent is reduced as compared to the mobile phase conductivity and the pH is substantially equivalent to the mobile phase pH; and, optionally, recovering at least one target compound or a purified liquid.

### CROSS-REFERENCE TO RELATED APPLICATIONS

This application is a filing under 35 U.S.C. § 371 and claims priority to international patent application number PCT/SE2006/001369 filed Nov. 30, 2006, published on Jun. 7, 2007, as WO 2007/064281, which claims priority to patent application number 0502683-6 filed in Sweden on Dec. 2, 2005.

### FIELD OF THE INVENTION

The present invention relates to the field of separation of bio-molecules from liquids, such as antibody purification, using a separation matrix which is especially suitable for purification of antibodies.

### BACKGROUND OF THE INVENTION

The immune system is composed of many interdependent cell types that collectively protect the body from bacterial, parasitic, fungal, viral infections and from the growth of tumour cells. The guards of the immune system are macrophages that continually roam the bloodstream of their host. When challenged by infection or immunisation, macrophages respond by engulfing invaders marked with foreign molecules known as antigens. This event, mediated by helper T cells, sets forth a complicated chain of responses that result in the stimulation of B-cells. These B-cells, in turn, produce proteins called antibodies, which bind to the foreign invader. The binding event between antibody and antigen marks the foreign invader for destruction via phagocytosis or activation of the complement system. Five different classes of antibodies, or immunoglobulins, exist: IgA, IgD, IgE, IgG, and IgM. They differ not only in their physiological roles but also in their structures. From a structural point of view, IgG antibodies are a particular class of immunoglobulins that have been extensively studied, perhaps because of the dominant role they play in a mature immune response.

The biological activity, which the immunoglobulins possess, is today exploited in a range of different applications in the human and veterinary diagnostic, health care and therapeutic sector. In fact, in the last few years, monoclonal antibodies and recombinant antibody constructs have become the largest class of proteins currently investigated in clinical trials and receiving FDA approval as therapeutics and diagnostics. Complementary to expression systems and production strategies, purification protocols are designed to obtain highly pure antibodies in a simple and cost-efficient manner. Traditional methods for isolation of immunoglobulins are based on selective reversible precipitation of the protein fraction comprising the immunoglobulins while leaving other groups of proteins in solution. Typical precipitation agents being ethanol, polyethylene glycol, lyotropic i.e. anti-chaotropic salts such as ammonium sulphate and potassium phosphate, and caprylic acid. Typically, these precipitation methods are giving very impure products while at the same time being time consuming and laborious. Furthermore, the addition of the precipitating agent to the raw material makes it difficult to use the supernatant for other purposes and creates a disposal problem, which is particularly relevant when speaking of large-scale purification of immunoglobulins.

Ion exchange chromatography is another well-known method of protein fractionation frequently used for isolation of immunoglobulins. However, since the charged ion exchange ligands will react with all oppositely charged compounds, the selectivity of ion exchange chromatography may be somewhat lower than other chromatographic separations.

Protein A and Protein G affinity chromatography are popular and widespread methods for isolation and purification of immunoglobulins, particularly for isolation of monoclonal antibodies, mainly due to the ease of use and the high purity obtained. Used in combination with ion exchange, hydrophobic interaction, hydroxyapatite and/or gel filtration steps, especially protein A-based methods have become the antibody purification method of choice for many biopharmaceutical companies. However, despite their common usage, there is a growing need and demand for effective alternatives addressing familiar problems associated with protein A-based media, such as cost, leakage and instability at increased pH values.

Hydrophobic interaction chromatography (HIC) is also a method widely described for isolation of immunoglobulins. However, hydrophobic matrices require an addition of lyotropic salts to the raw material to make the immunoglobulin bind efficiently. The bound antibody is released from the matrix by lowering the concentration of lyotropic salt in a continuous or stepwise gradient. If a highly pure product is the object, it is recommended to combine the hydrophobic chromatography with a further step. Thus, a disadvantage of this procedure is the necessity to add lyotropic salt to the raw material as this may cause potential problems, and thereby an increased cost to the large-scale user. For other raw materials than cell culture supernatants such as whey, plasma, and egg yolk the addition of lyotropic salts to the raw materials would in many instances be prohibitive in large-scale applications as the salt could prevent any economically feasible use of the immunoglobulin depleted raw material. An additional problem in large-scale applications would be the disposal of several thousand litres of waste.

Thiophilic adsorption chromatography was introduced by J. Porath in 1985 (J. Porath et al; FEBS Letters, vol. 185, p.306, 1985) as a new chromatographic adsorption principle for isolation of immunoglobulins. In this paper, it is described how divinyl sulphone activated agarose coupled with various ligands comprising a free mercapto-group show specific binding of immunoglobulins in the presence of 0.5 M potassium sulphate, i.e. a lyotropic salt. It was postulated that the sulphone group, from the vinyl sulphone spacer, and the resulting thioether in the ligand was a structural necessity to obtain the described specificity and capacity for binding of antibodies. It was however later shown that the thioether could be replaced by nitrogen or oxygen if the ligand further comprised an aromatic radical (K. L. Knudsen et al, Analytical Biochemistry, vol. 201, p. 170, 1992). Although the matrices described for thiophilic chromatography generally show good performance, they also have a major disadvantage in that it is needed to add lyotropic salts to the raw material to ensure efficient binding of the immunoglobulin, which is a problem for the reasons discussed above.

Other thiophilic ligands coupled to epoxy activated agarose have been disclosed in (J. Porath et. al. Makromol. Chem., Makromol. Symp., vol. 17, p.359, 1988) and (A. Schwarz et. al., Journal of Chromatography B, vol. 664, pp. 83-88, 1995), e.g. 2-mercaptopyridine, 2-mercaptopyrimidine, and 2-mercaptothiazoline. However, all these affinity matrices still have inadequate affinity constants to ensure an efficient binding of the antibody without added lyotropic salts.

U.S. Pat. No. 6,498,236 (Upfront Chromatography) relates to isolation of immunoglobulins. The method disclosed involves the steps of contacting a solution that comprises a negatively charged detergent and contains immunoglobulin(s) with a solid phase matrix, whereby at least a part of the immunoglobulins becomes bound to the solid phase matrix; and contacting the solid phase matrix with an eluent in order to liberate the immunoglobulin(s) from the solid phase matrix. The immunoglobulin-containing solution is further characterised by having a pH in the range of 2.0 to 10.0, a total salt content corresponding to an ionic strength of at the most 2.0, and lyotropic salts in a concentration of at the most 0.4 M. The detergent present in the solution is believed to suppress the adherence of other biomolecules to the matrix, and is exemplified by octyl sulphate, bromphenol blue, octane sulphonate, sodium laurylsarcosinate, and hexane sulphonate. The solid phase matrix is defined by the formula M-SP1-L, wherein M designates the matrix backbone; SP1 designates a spacer; and L designates a ligand comprising a mono- or bicyclic optionally substituted aromatic or heteroaromatic moiety.

Liu et al (Yang Liu, Rui Zhao, Dihua Shangguan, Hongwu Zhang, Guoquan Liu: Novel sulphmethazine ligand used for one-step purification of immunoglobulin G from human plasma, Journal of Chromatography B, 792 (2003) 177-185) investigated the affinity of sulphmethazin (SMZ) to human IgG. Thus, a ligand is disclosed, which comprises a sulphonyl group wherein the R group is a heterocyclic ring. According to this article, SMZ was immobilised on monodisperse, non-porous, cross-linked poly(glycidyl methacrylate) beads. The beads were then used in high-performance affinity chromatography for isolation of IgG from human plasma. Maximal adsorption was achieved at pH 5.5. The beads presented minimal non-specific interaction with other proteins. Thus, the ligands were capable of adsorbing antibodies, while their interaction with other proteins was just sufficient to provide retardation thereof in the adsorption buffer used. However, as is well known, ester compounds such as methacrylate are easily hydrolysed at increased pH values. Consequently, similar to Protein A and Protein G matrices, the therein disclosed separation matrix would be expected to be unstable at the commonly used cleaning in place (cip) procedures.

U.S. Pat. No. 4,725,355 (Terumo Kabushiki Kaisha) relates to a body fluid purification medium and apparatus, and more specifically to a support having an adsorbent fixed thereto for use to remove pathogenic substances such as plasma proteins in a body fluid. According to U.S. Pat. No. 4,725,355, in order to perform extracorporeal blood purification therapy when treating a patient, it is preferable that a pathogenic substance be eliminated at a still higher efficiency and adverse influences on the blood be extremely small. The adsorbent provided according to U.S. Pat. No. 4,725,355 includes at least one sulfa drug. According to U.S. Pat. No. 4,725,355, the azole ring in a sulfa drug exhibits a hydrophobic property, while the hetero atom in the ring has a lone pair of electrons and serves as a protein acceptor. The sulphonamide portion of the sulfa drug is stated to have hydrogen bondability.

WO 2005/061543 (Amersham Biosciences AB) relates to the purification of immunoglobulins. More specifically, WO 2005/061543 discloses a novel separation matrix comprised of a solid support to which ligands have been immobilised, wherein said ligands comprise at least one aliphatic sulphonamide. A method of isolating antibodies is also disclosed, which method comprises the steps of adsorbing antibodies to the above-described separation matrix and releasing the antibodies by passing an eluent over said matrix. It is stated in general terms that the salt concentration can be optimised for each specific ligand structure; and in one embodiment the adsorption is provided at a salt concentration of about 0.25 M NaSa₂SO₄ while in another embodiment, the adsorption is allegedly taking place at a salt concentration above about 0.5 M Na₂SO₄.

The release of adsorbed antibodies may be provided by any one of a number of well known elution schemes, such as by decreasing salt concentration; by increase or decrease of pH; by adding a competitive binder; or by displacement. However, despite the general statements, the Experimental part of WO 2005/061543 shows some more specific observations. For example, if the salt content in the adsorption buffer increases, the selectivity for IgG is stated to decrease as a result of adsorption of the other proteins. Further, in some cases a pH decrease was necessary to release IgG adsorbed at 0.25 M Na2SO4.

Thus, even though WO 2005/061543 describes a novel separation matrix, work appears to remain to develop the most advantageous conditions for the use thereof.

Finally, SE 2005/001002 (Amersham Biosciences), published as WO2006/001771, relates to a separation matrix comprised of aromatic sulphonamide ligands coupled to a support. The disclosed separation matrix is advantageously used for the purification of antibodies.

Thus, there is still a need of alternative methods for purification of antibodies or antibody constructs, which observe the demands of purity, safety, potency and cost effectiveness.

### SUMMARY OF THE INVENTION

One aspect of the present invention is to provide a new ligands and a method of hydrophobic interaction chromatography (HIC) using the same. This can be achieved by a method as defined in claim 1, wherein sulphonamide ligands are used under HIC conditions.

Another aspect of the present invention is to provide a method of hydrophobic interaction chromatography (HIC), which provides different selectivities as compared to conventional HIC media.

Yet another aspect of the invention is to provide a method of isolating target compounds from a liquid by adsorption thereof to a separation matrix, which method does not require any addition of detergent to achieve adsorption.

A specific aspect of the invention is a method as above, which is used for the isolation of one or more antibodies.

Further aspects and advantages of the invention will appear from the detailed description that follows.

### BRIEF DESCRIPTION OF THE DRAWINGS

FIG. 1 shows the respective ligand structures of the prototype separation matrix Butyl sulphonamide SEPHAROSE™ 6 Fast Flow; and Butyl SEPHAROSE™ 4 Fast Flow.

FIG. 2 shows the chromatographic retardation of human immunoglobulin on Butyl sulphonamide SEPHAROSE™ Fast Flow.

FIG. 3 shows the retardation of human immunoglobulin on Butyl SEPHAROSE™ 4 Fast Flow.

FIG. 4 shows the retardation of α-chymotrypsinogen, lysozyme, ribonuclease A and cytochrome C on Butyl sulphonamide SEPHAROSE™ 6 Fast Flow.

FIG. 5 shows the retardation of α-chymotrypsinogen, lysozyme, ribonuclease A and cytochrome C on Butyl SEPHAROSE™ Fast Flow.

FIG. 6 shows the respective ligand structures of the prototype separation matrix Octyl sulphonamide SEPHAROSE™ 6 Fast Flow; and of the commercial product Octyl SEPHAROSE™ 4 Fast Flow.

FIG. 7 shows the chromatographic retardation of human immunoglobulin on Octyl sulphonamide SEPHAROSE™ 6 Fast Flow.

FIG. 8 shows the retardation of human immunoglobulin on Octyl SEPHAROSE™ 4 Fast Flow.

FIG. 9 shows the retardation of α-chymotrypsinogen, lysozyme, ribonuclease A and cytochrome C on Octyl sulphonamide SEPHAROSE™ 6 Fast Flow.

FIG. 10 shows the retardation of α-chymotrypsinogen, lysozyme, ribonuclease A and cytochrome C on Octyl SEPHAROSE™ 4 Fast Flow

FIG. 11 shows the retardation of human immunoglobulin on Octyl sulphonamide SEPHAROSE™ 6 Fast Flow.

FIG. 12 shows the ligand structure of the prototype Pentaethylene methyl/butyl sulphonamide SEPHAROSE™ 6 Fast Flow.

FIG. 13 shows the chromatographic retardation of human immunoglobulin on Pentaethylene methyl/butyl (80/20) sulphonamide SEPHAROSE™ 6 Fast Flow.

FIG. 14 shows the retardation of α-chymotrypsinogen, lysozyme, ribonuclease A and cytochrome C on Pentaethylene methyl/butyl (80/20) sulphonamide SEPHAROSE™ 6 Fast Flow.

FIG. 15 shows the ligand structure of Phenyl sulphonamide SEPHAROSE™ 6 Fast Flow.

FIG. 16 shows the retardation of human immunoglobulin on Phenyl sulphonamide SEPHAROSE™ 6 Fast Flow.

FIG. 17 shows the retardation of α-chymotrypsinogen, lysozyme, ribonuclease A and cytochrome C on Phenyl sulphonamide SEPHAROSE™ 6 Fast Flow.

### DEFINITIONS

The term “hydrophobic interaction chromatography” refers to a method of separating a target based on the strength of its relative hydrophobic interactions with a hydrophobic separation matrix. In this context, “hydrophobicity” is defined as the repulsion between a non-polar compound and a polar environment.

The terms “antibody” and “immunoglobulin” are used herein interchangeably.

The term “sulphonamide” is used in its conventional meaning i.e. as a chemical compound comprising one or more amides of sulphonic acids.

A “sulphonyl group” is defined by formula —S(═O)₂R, wherein R denotes an organic group.

The term “aromatic sulphonamide” refers to a sulphonamide wherein the R group comprises one or more aromatic groups.

The term “aromatic” group refers to a group, wherein the number of π electrons can be calculated according to Huckels rule: (4n+2), wherein n is a positive integer or zero.

The term “bicyclic” and “tricyclic” means that the residue comprises two or three rings, respectively. Said rings may be fused rings or separate rings. Likewise, a residue comprising any further number of rings can be comprised of fused or separate rings.

The term “protonatable” group means a group capable of adding a hydrogen.

The term “aliphatic sulphonamide” refers to a sulphonamide wherein at least one R group comprises an aliphatic group.

The term “aliphatic” group embraces linear and branched groups; as well as cyclic and acyclic groups.

A “primary amine” is defined by formula RNH₂, wherein R denotes an organic group.

A “secondary amine” is defined by formula R′₂NH, wherein R′ denotes an organic group.

The term “ligand” means herein molecules or compounds capable of interaction with target compounds, such as antibodies.

The term “spacer arm” means herein an element that distances a ligand from the support of a separation matrix.

The term “surface” when used in the context of a porous support embraces the pore surfaces as well as to the actual outer surfaces.

The term “mobile phase” is used herein interchangeably with “adsorption buffer”.

The term “eluent” is used in its conventional meaning in this field, i.e. a buffer of suitable pH and/or ionic strength to release one or more compounds from a separation matrix.

The term “gradient elution” means gradually changing the conditions from binding to non-binding conditions.

### DETAILED DESCRIPTION OF THE INVENTION

Thus, as appears from the above, the present invention relates to a new field within hydrophobic interaction chromatography (HIC). In a first aspect, the invention relates to a method of isolating at least one target compound from a liquid, which method comprises the steps of
(a) providing a mobile phase, which comprises at least one target compound and wherein the conductivity corresponds to ≧0.6 M (NH₄)₂SO₄;
(b) contacting said mobile phase with a separation matrix comprising one or more sulphonamide groups to adsorb one or more target compounds;
(c) contacting an eluent with said matrix to release one or more target compounds, wherein the conductivity of the eluent is reduced as compared to the mobile phase conductivity; and the pH is substantially equivalent to the mobile phase pH; and, optionally,
(d) recovering at least one target compound. Alternatively, a purified liquid is recovered.

The target compound(s) are preferably recovered from a fraction of the eluent. The pH of the mobile phase is controlled by adding a suitable buffer, the pH of which may be anywhere in the range of 2-12. In one embodiment, the pH of the mobile phase is buffered to close to neutral, such as 6.5-8.3, and more specifically 7.2-7.6, e.g. about 7.4. Thus, in an advantageous embodiment, the pH of the mobile phase is in the range of 6-8 during the contact with the separation matrix and adsorption of target compounds. The mobile phase may be comprised of any suitable buffer of the appropriate pH, such as phosphate buffer. In a specific embodiment, the mobile phase comprises TRIS buffer.

As appears from the above, the present invention relates to the use of sulphonamide ligands under conditions conventional known as HIC conditions. Thus, the adsorption is carried out under relatively high conductivities. For simplicity, in the present context, it is understood that the salt concentration as discussed will give an estimate of the conductivity of a liquid or buffer. Conductivity is easily measured using well known methods and commercially available detectors. Thus, in one embodiment, the conductivity of the mobile phase corresponds to ≧0.6 M, such as ≧0.8 M, ≧1.0 M or ≧1.5 M, and preferably ≧2.0 M (NH₄)₂SO₄.

The conductivity of the liquids (mobile phase and eluent) is discussed herein in values corresponding to a certain concentration of ammonium sulphate, but the term “conductivity corresponding to a specified concentration of (NH₄)₂SO₄” is understood to include other salt(s), optionally in slightly different concentrations, which provide(s) substantially the same conductivity as the specified range of (NH₄)₂SO₄. Thus, the present invention embraces the use of any salt that provides the conductivity defined, such as other ammonium or sodium salt(s), e.g. sulphate(s) or phosphate(s). As is easily understood, two or more salts can also be mixed to provide the desired conductivity. In a specific embodiment, the mobile phase comprises an ammonium salt. In a specific embodiment, in step (a) a mobile phase is provided, which comprises at least one target compound and ≧0.6 M (NH₄)₂SO₄. Further, step (a) embraces an embodiment wherein the conductivity is already within the appropriate range when contacted with the sample. In an alternative embodiment, the conductivity is adjusted to the appropriate range by addition of on or more suitable salts.

The elution of adsorbed target compound(s) is carried out following the principles of conventional HIC, such as by decreasing the salt concentration and/or by adding an organic solvent.

Thus, in a first embodiment of the elution, the adsorbed compounds are released from the separation matrix by reducing the salt concentration. This can be achieved by adding an eluent of a lower conductivity, which can range from a value just below the adsorption conductivity to zero. In an advantageous embodiment, step (c) uses a decreasing salt gradient elution. The gradient may be continuous or stepwise. In an alternative embodiment, which is advantageously used in an already optimised purification protocol, the elution is carried out by adding an eluent of a set conductivity.

The pH of the eluent is substantially equivalent to the mobile phase pH, as is common in HIC. As the skilled person will realise, small variations may occur such as caused e.g. by increasing the salt concentration. In this embodiment of the invention, the release of the adsorbed target compound(s) is predominantly caused by the reduced conductivity.

In a second embodiment of the elution, adsorbed target compound(s) are released by adding an eluent comprising organic solvent, which is also a well known HIC elution principle.

Thus, one embodiment of the invention is a method of isolating at least one target compound from a liquid, which method comprises the steps of
(a) providing a mobile phase, which comprises at least one target compound and wherein the conductivity corresponds to ≧0.6 M (NH₄)₂SO₄;
(b) contacting said mobile phase with a separation matrix comprising one or more sulphonamide groups to adsorb one or more target compounds;
(c) contacting an eluent with said matrix to release one or more target compounds, wherein the eluent comprises at least one organic solvent; and, optionally,
(d) recovering at least one target compound. Alternatively, a purified liquid is recovered.

The conditions other than the eluent are preferably as discussed above. In this embodiment, the eluent may comprise any organic solvents, such as ethanol or isopropanol. In a specific embodiment, the eluent comprises a decreasing salt gradient in addition to the organic solvent(s).

In a first embodiment of the present method, the separation matrix is comprised of a support to which one or more aliphatic sulphonamide groups have been immobilised as ligands, optionally via spacer arms. As is well known, a sulphonamide is comprised of an amine, wherein at least one of the R groups of said amine is a sulphonyl group. Thus, in this embodiment, the R group of the sulphonyl is an aliphatic acyclic or cyclic group, such as a linear chain of 1-8 carbon atoms and/or heteroatoms. The aliphatic groups may be substituted.

In one embodiment, the R group comprises a linear chain of 1-4 carbon atoms and/or heteroatoms. In one embodiment, the aliphatic R group of the sulphonyl comprises a methyl group. In an alternative embodiment, the aliphatic R group of the sulphonyl comprises an ethyl group.

In another embodiment, the R group comprises a linear chain of 4-8, such as 5-9, carbon atoms and/or heteroatoms. In one embodiment, the aliphatic R group of the sulphonyl comprises a butyl group. In an alternative embodiment, the aliphatic R group of the sulphonyl comprises an octyl group.

In a further embodiment, the R group of the sulphonyl is one or more F atoms.

In a second embodiment of the present method, the separation matrix is comprised of a support to which one or more aromatic sulphonamide groups have been immobilised as ligands, optionally via spacer arms. In a specific embodiment, the ligands are substantially devoid of protonatable groups. In this context, the term “substantially devoid of protonatable groups” is understood to mean that no such groups constitute part of the ligand, and hence that the interaction with a target molecule does not involve protonatable groups to any substantial extent.

Further, in one embodiment of the method using aromatic sulphonamide ligands, the R group of the sulphonyl is a substituted or unsubstituted aromatic or heteroaromatic group, such as a mono- or polyaromatic group. More specifically, the R group may e.g. be monocyclic, bicyclic or tricyclic. Examples of aromatic residues are phenyl; benzyl; benzoyl; naphtyl; and tosyl. Heteroaromatic groups may comprise one or more of the heteroatoms N, O and S, and may be exemplified e.g. by thienyl; furyl; and pyridyl. In one embodiment, the substituents are electron withdrawing. The substituents may be single atoms, such as halogens or carbon atoms; or groups, such as —N(O)₂. The substituents may alternatively be linear or branched carbon chains.

In a third embodiment of the present method, the separation contains both aromatic and aliphatic sulphonamides. The sulphonamide ligand may comprise further substituents. As the skilled person in this field will realise, the nature of the substituents may be utilised to enhance the binding properties of the ligands. However, it is also understood that the nature and size of the ligand, especially of the R group and its substituents, should be selected so as not to inhibit, e.g. by steric hindrance, the binding of a target molecule, such as an antibody.

In one embodiment of the method, the nitrogen of the sulphonamide groups is derived from a primary and/or secondary amine.

In another embodiment of the method, the ligands comprise sulphonylated monoamines, such as cysteamine or ammonia. In an alternative embodiment, the ligands are sulphonylated polyamines. Such sulphonylated polyamines may comprise any convenient number of amines, such as 2-10. In an illustrative embodiment, each polyamine comprises 2-6 amines. In another embodiment, the ligands comprise more than one sulphonyl group. Such further sulphonyl groups may be part of the R group of the sulphonamide; and/or form a part of a spacer arm that connects the ligand with the support.

The ligands used in the present method are easily prepared by the skilled person in this field according to standard methods, see e.g. WO 2005/061543 (Amersham Biosciences), which is hereby incorporated herein via reference. In one embodiment of the method, some, such as about 50%, more specifically about 75% and especially about 90%, or substantially all of the sulphonamide groups have been coupled to the support via sulphur. In an alternative embodiment, some, such as about 50%, more specifically about 75% and especially about 90%, or substantially all of the sulphonamide groups have been coupled to the support via nitrogen.

In a specific embodiment of the present separation matrix, the ligands are present as units of a polymer immobilised to the support, such as repetitive or randomly positioned units. The polymer may be any suitable polyamine, such as polyalkyleneimine. In one embodiment, the polymer is a polyethylene amine. As the skilled person in this field will realise, the amine content of such a polymer may be varied, e.g. to comprise primary and/or secondary amines in any desired order. Thus, in one embodiment, the polymer exhibit two or more different ligand groups. The polymers are easily produced from suitable monomers according standard methods in this field. Methods of coupling the polyamines to a support are also well known and easily performed by the skilled person in this field, for example by in situ polymerisation or grafting of polymers, see e.g. PCT/SE02/02159, which published as WO2003/046063 (Ihre et al).

In addition to the sulphonamide ligands, the separation matrix used in the present invention may comprise one or more other functional groups. Thus, in one embodiment, the ligands of the separation matrix are multi-modal ligands in the sense that they are capable of interacting with the target using two or more functions. The additional or secondary functional group can be easily introduced, for example via the introduction of different substituents on the sulphonamide group, or via a spacer, or by alkylation of the nitrogen atom of the sulphonamide group or simply by a stochastic approach by introducing new ligands (two or more different ligand structures) on the above-described sulphonamide matrix. Additional functional groups are, for example, selected from the group consisting of aromatic group; heterocyclic and aliphatic groups; H-bond donor and acceptor-containing groups; chargeable functional groups such as amines and acidic groups; poly hydroxylated groups such as dextran; polyethylene glycol derivatives; and fluorine atom-containing groups.

A specific aspect of the invention is a method for isolation of at least one target compound, which uses a separation matrix comprised of one or more of the above-discussed sulphonamide ligands as well as charge-inducible groups. Thus, in this aspect, a pH change may be utilised to release adsorbed target compounds by electrostatic repulsion. Charge-inducible groups are well known to the skilled person in this field.

Thus, in one embodiment, the present method uses a porous support onto which ligands selected from the group consisting of hydrophobic interaction chromatography (HIC) groups; ion exchange groups; affinity groups; and metal chelating groups; have been immobilised.

In a specific aspect, the present invention relates to a novel group of sulphonamide ligands, wherein the sulphonamide group(s) comprise longer R groups than previously suggested, such as 4-8 carbon atoms and/or heteroatoms. Advantageously, the novel ligands are comprised of one or more sulphonamide groups and one or more additional groups, which substantially lack affinity of water. In one embodiment, the novel sulphonamide ligand is butyl sulphonamide. In another embodiment, the novel sulphonamide ligand is octyl sulphonamide. The novel ligands may be immobilised to any one of the herein discussed supports to provide novel separation matrices.

In one embodiment, the sulphonamide ligands used in the present method are immobilised to the support via extenders, or a coating polymer layer. Such extenders, which are well known and also known as “flexible arms”, may be organic or synthetic polymers. Thus, the support may e.g. be coated with dextran extenders, to provide a hydrophilic nature to the support, to which the ligands are immobilised according to well known methods in this field.

The porous support of the present separation matrix may be of any suitable material. In one embodiment, the support is comprised of a cross-linked carbohydrate material, such as agarose, agar, cellulose, dextran, chitosan, konjac, carrageenan, gellan, alginate etc. The support can easily be prepared according to standard methods, such as inverse suspension gelation (S Hjertén: Biochim Biophys Acta 79(2), 393-398 (1964). Alternatively, the support is a commercially available product, such as SEPHAROSE™ FF (Amersham Biosciences AB, Uppsala, Sweden). Thus, in one embodiment of the present matrix, the support is a cross-linked polysaccharide. In a specific embodiment, said polysaccharide is agarose. Such carbohydrate materials are commonly allylated before immobilisation of ligands thereof. In brief, allylation can be carried out with allyl glycidyl ether, allyl bromide or any other suitable activation agent following standard methods.

In an alternative embodiment, the support used in the present method is comprised of cross-linked synthetic polymers, such as styrene or styrene derivatives, divinylbenzene, acrylamides, acrylate esters, methacrylate esters, vinyl esters, vinyl amides etc. Supports of such polymers are easily produced according to standard methods, see e.g. “Styrene based polymer supports developed by suspension polymerization” (R Arshady: Chimica e L'Industria 70(9), 70-75 (1988)). Alternatively, a commercially available product, such as Source™ (Amersham Biosciences AB, Uppsala, Sweden) can be surface-modified according to the invention. However, in this embodiment, the surface of the support is preferably modified to modify its hydrobicity, usually be converting the some of the exposed residual double bonds to hydroxyl groups.

The present method can use a separation matrix in any suitable form, such as a chromatography matrix, e.g. in the form of essentially spherical particles or a monolith; a filter or membrane; a chip or the like. Thus, in an advantageous embodiment, the separation matrix is provided in a chromatography column, such as a packed column. The column may be of a size suitable for laboratory scale or large-scale purification of target compounds.

As will be shown in the experimental part below, in general, the present method provides isolation of target compounds at selectivities that differ from the use of conventional reference HIC media. The skilled person in this field can easily select the optimal conditions for each sulphonamide ligand structure using routine experimentation. Further, it is well known in this field that the properties of a separation matrix can be optimised by variation of either the nature of the ligands, in this case, the R group of the sulphonamide, or the degree of substitution i.e. the ligand density on the support. Even though as a general rule, the present invention uses a higher conductivity at the adsorption step than the prior art, the salt concentration and the kind of salt in the adsorption buffer can be further optimised for each ligand.

The target compound isolated according to the invention may be a desired product; or a compound which it is desired to remove in order to purify a liquid. The target compound may be any compound or molecule, such as a biomolecule, an organic molecule or an inorganic molecule. In this context, it is understood that the term “compound” embraces any chemical entity; molecule or species. Thus, in one embodiment, the target molecule is selected from the group consisting of proteins, such as polypeptides and antibodies; nucleic acids, such as DNA, e.g. plasmids, RNA, and oligonucleotides; cells, such as prokaryotic or eukaryotic cells; virus, such as retrovirus, influenza virus and adenovirus; toxins; polysaccharides; lipids; and organic drug candidates. It is understood that the term “target compound” also embraces any fragments of the above.

In one embodiment of the present method, antibodies are purified from a liquid comprising one or more other proteins. In this context, it is to be understood that the term “antibodies” also includes antibody fragments and any fusion protein that comprises an antibody or an antibody fragment. Thus, the present method is useful to isolate any immunoglobulin-like molecule, which presents the binding properties of an antibody. The liquid comprising an antibody may for example be a liquid originating from a cell culture producing antibodies or a fermentation broth, from which it is desired to purify one or more desired antibodies. Alternatively, the liquid may be blood or blood plasma, from which it is desired to remove one or more antibodies to obtain a liquid which is pure in that respect.

The present method is useful to recover any kind of monoclonal or polyclonal antibody, such as antibodies originating from mammalian hosts, such as mice, rodents, primates and humans, or antibodies originating from cultured cells such as hybridoma cells. In one embodiment, the antibodies recovered in step (d) are human or humanised antibodies. The antibodies may be of any class, i.e. selected from the group that consists of IgA, IgD, IgE, IgG, and IgM. In a specific embodiment, the antibodies recovered in step (d) are immunoglobulin G (IgG). The present invention also encompasses the purification of fragments of any one of the above mentioned antibodies as well as fusion proteins comprising such antibodies. The isolated or purified target molecules may be useful in the medical field as antibody drugs; for example in personalised medicine where a specific drug is designed for each individual in need.

The present method is useful as one step in a purification protocol, such as one step in the chromatographic purification of proteins, such as antibodies. Thus, the method may be a first step, known as the capture step; an intermediate step; or a polishing step, wherein the final impurities are removed. Thus, the present method may be combined by one or more other chromatography steps, such as affinity chromatography using e.g. Protein A media.

In a specific one embodiment, the present method encompasses a method as defined above and in addition a subsequent determination of the amount of target compound, such as protein or antibody, spectrophotometrically. Such methods and useful equipment are well known to the skilled person in this field. The present is also useful in analytical procedures, and may provide a tool in the diagnostic field. Examples of fields where quantitative determination of proteins such as antibodies is useful are quality control of food products, environmental control, and monitoring and control of industrial processes.

In another aspect, the present invention relates to a kit for isolating at least one target compound from a liquid, which kit comprises, in separate compartments, a separation matrix comprising sulphonamide ligands; an adsorption buffer having a salt concentration of ≧0.8 M; and an eluent, wherein the eluent presents substantially the same pH value as the adsorption buffer and no salt. In an alternative embodiment, the kit comprises two or more different separation matrices. In a specific embodiment, the column is provided with luer adaptors, tubing connectors, and domed nuts and written instructions for purification of target compounds such as antibodies in separate compartments. In another embodiment, the kit comprises written instructions that teaches the use of the kit for antibody purification.

In a final aspect, the invention relates to the use of a separation matrix comprising one or more sulphonamide ligands as described above in hydrophobic interaction chromatography. Such use may e.g. be to purify target compounds, e.g. proteins, such as monoclonal or polyclonal antibodies; or, alternatively, to purify a liquid such as blood plasma, e.g. human blood plasma, from undesired target compounds such as polyclonal antibodies, e.g. IgG.

### DETAILED DESCRIPTION OF THE DRAWINGS

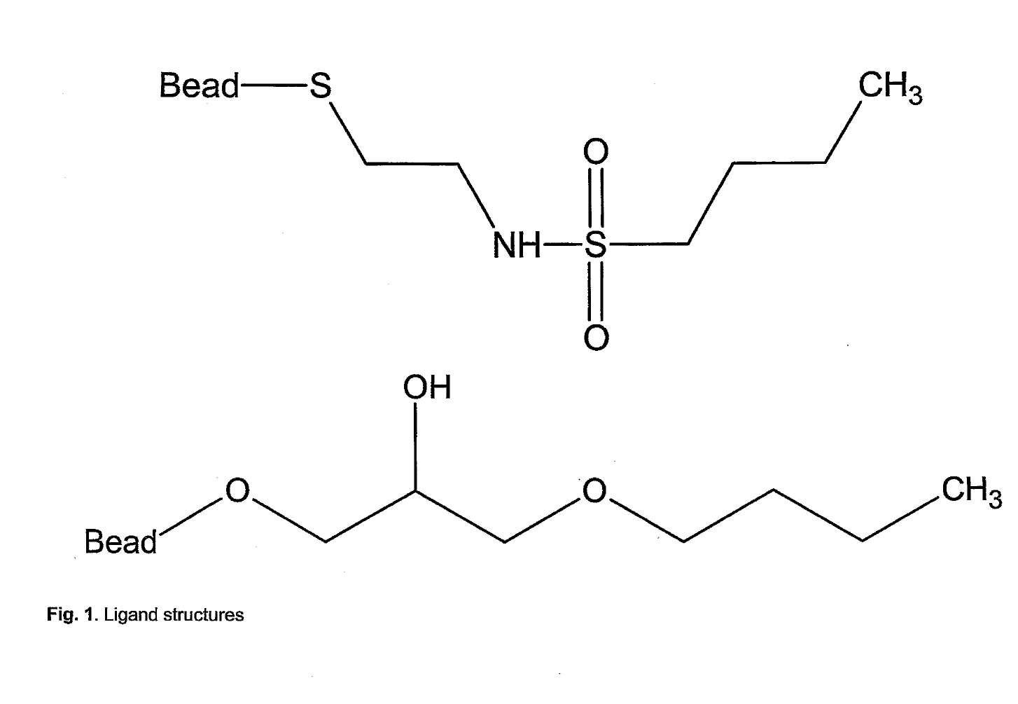

FIG. 1 shows the respective ligand structures of the prototype separation matrix Butyl sulphonamide SEPHAROSE™ 6 Fast Flow; and Butyl SEPHAROSE™ 4 Fast Flow, which is a commercial product available from GE Healthcare, Uppsala, Sweden.

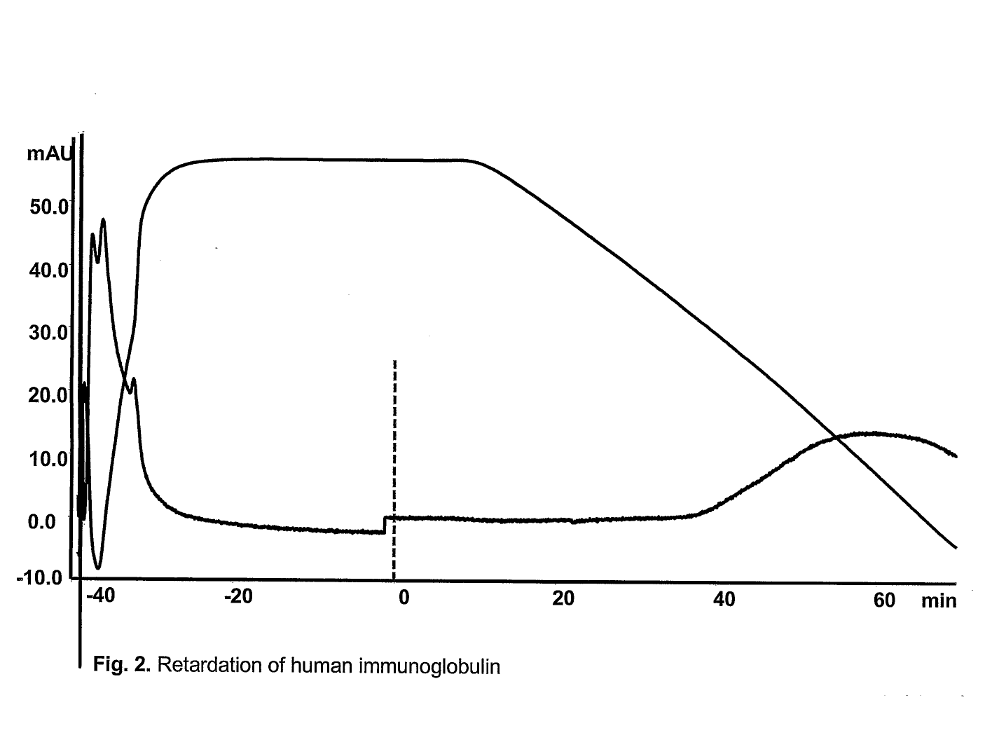

FIG. 2 shows the chromatographic retardation of human immunoglobulin on Butyl sulphonamide SEPHAROSE™ Fast Flow. 50 mM phosphate buffer (pH 7.0) with 0.80 M (NH₄)₂SO₄ was used as buffer A and 20 mM phosphate buffer (pH 7.0) as buffer B, as explained in Example 2 below.

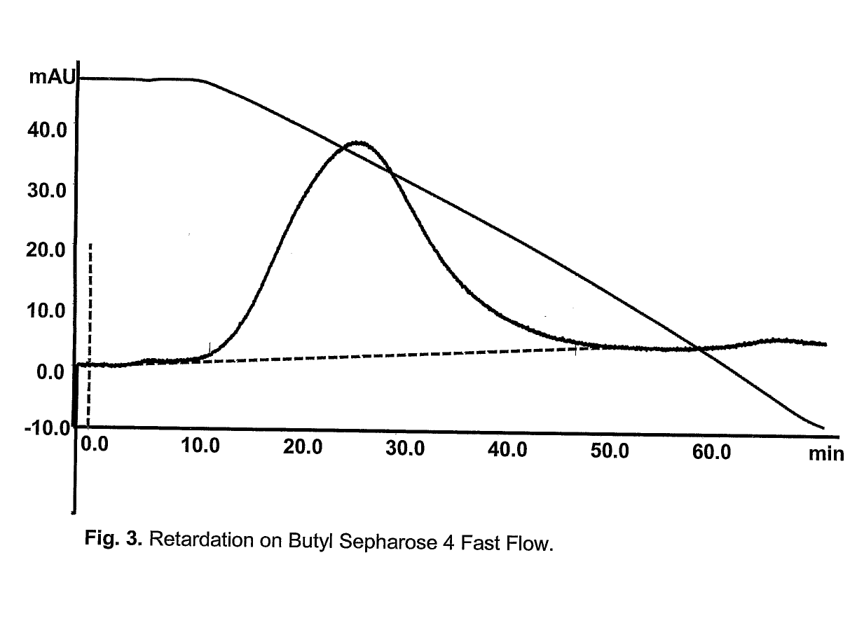

FIG. 3 shows the retardation of human immunoglobulin on Butyl SEPHAROSE™ 4 Fast Flow. 50 mM phosphate buffer (pH 7.0) with 0.80 M (NH₄)₂SO₄ was used as buffer A and 20 mM phosphate buffer (pH 7.0) as buffer B, as explained in Example 2 below.

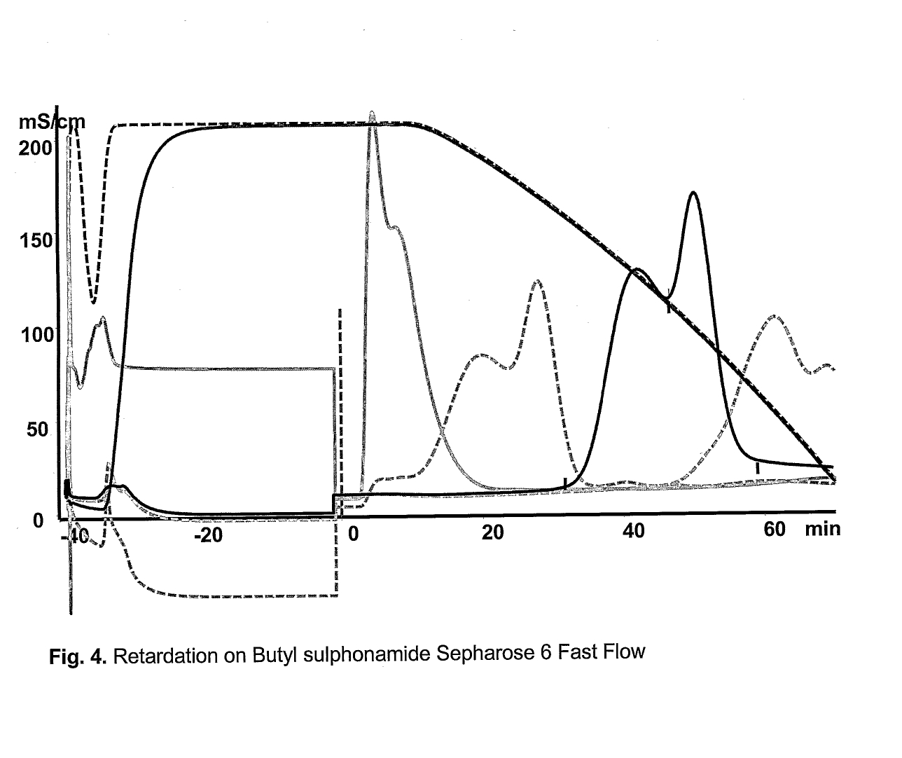

FIG. 4 shows the retardation of α-chymotrypsinogen, lysozyme, ribonuclease A and cytochrome C on Butyl sulphonamide SEPHAROSE™ 6 Fast Flow.

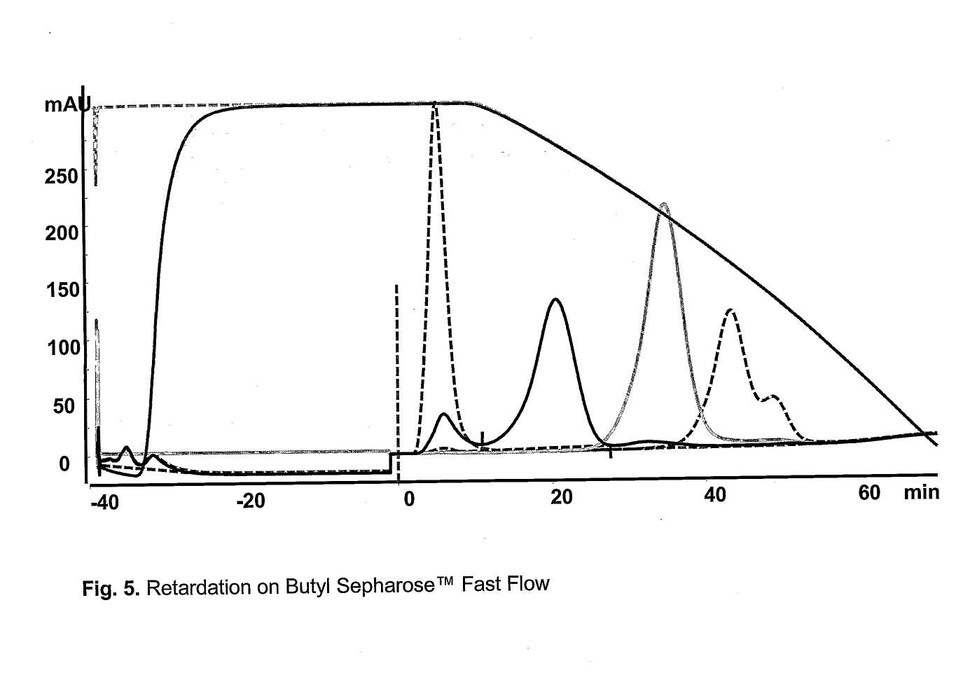

FIG. 5 shows the retardation of α-chymotrypsinogen, lysozyme, ribonuclease A and cytochrome C on Butyl SEPHAROSE™ Fast Flow.

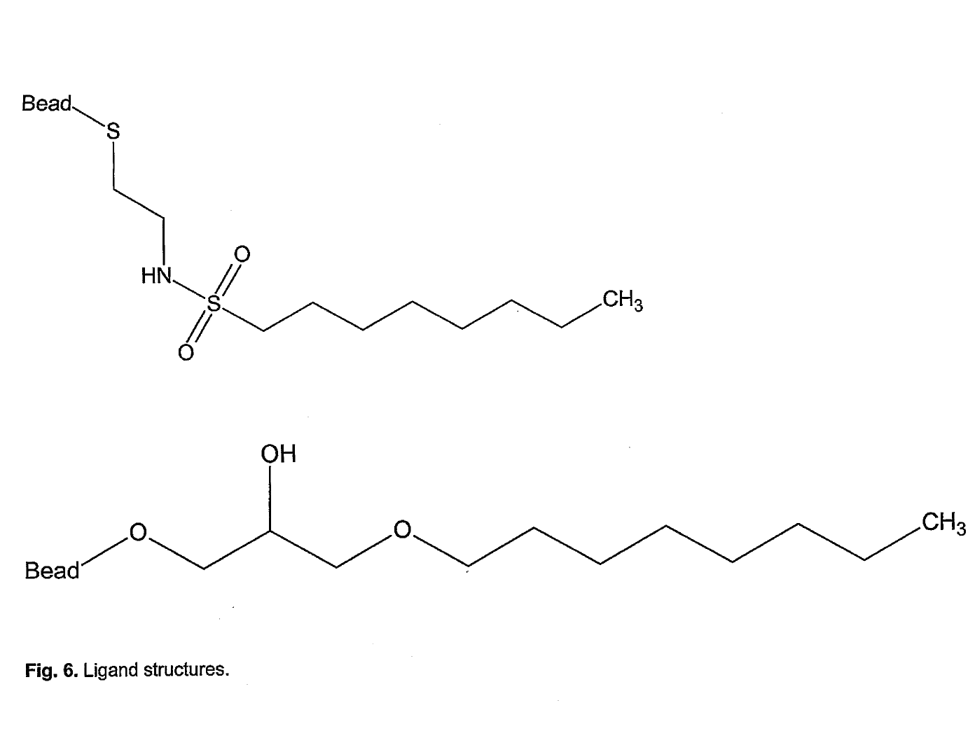

FIG. 6 shows the respective ligand structures of the prototype separation matrix Octyl sulphonamide SEPHAROSE™ 6 Fast Flow; and the commercial product Octyl SEPHAROSE™ 4 Fast Flow.

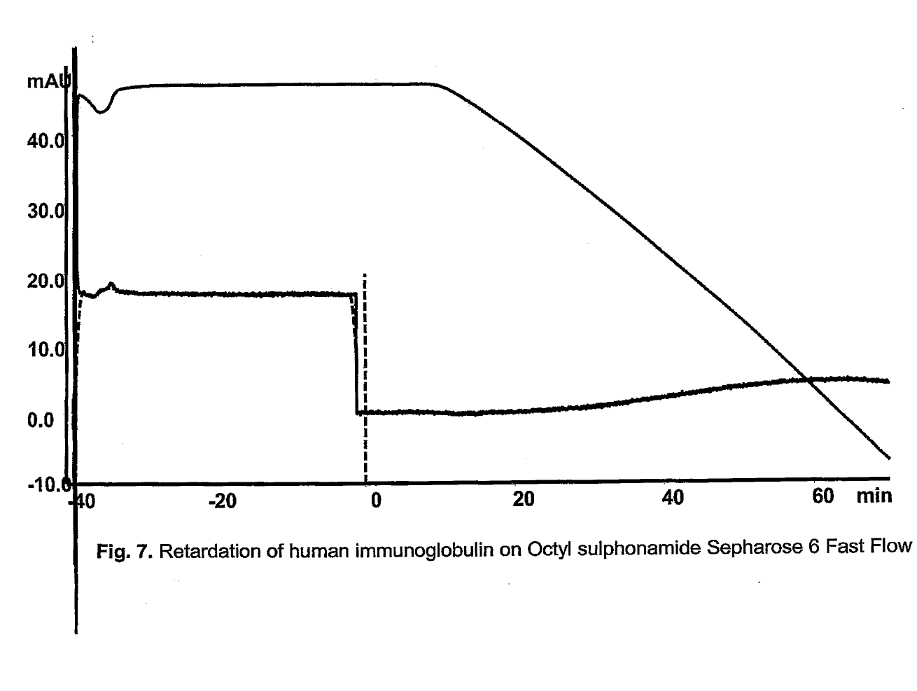

FIG. 7 shows the chromatographic retardation of human immunoglobulin on Octyl sulphonamide SEPHAROSE™ 6 Fast Flow. 50 mM phosphate buffer (pH 7.0) with 0.80 M (NH₄)₂SO₄ was used as buffer A and 20 mM phosphate buffer (pH 7.0) as buffer B (see the experimental part below).

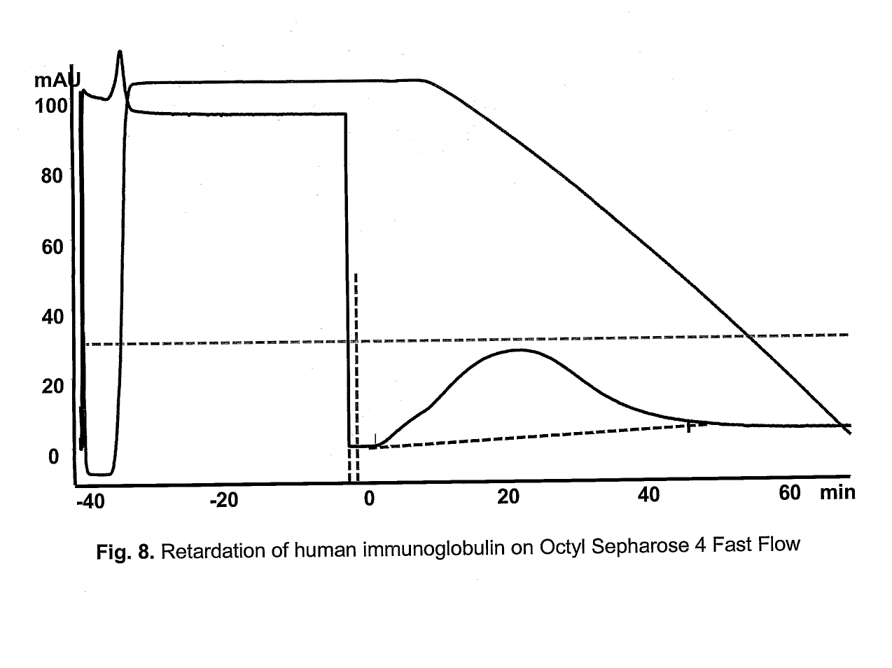

FIG. 8 shows the retardation of human immunoglobulin on Octyl SEPHAROSE™ 4 Fast Flow. 50 mM phosphate buffer (pH 7.0) with 0.80 M (NH₄)₂SO₄ was used as buffer A and 20 mM phosphate buffer (pH 7.0) as buffer B (see the experimental part below).

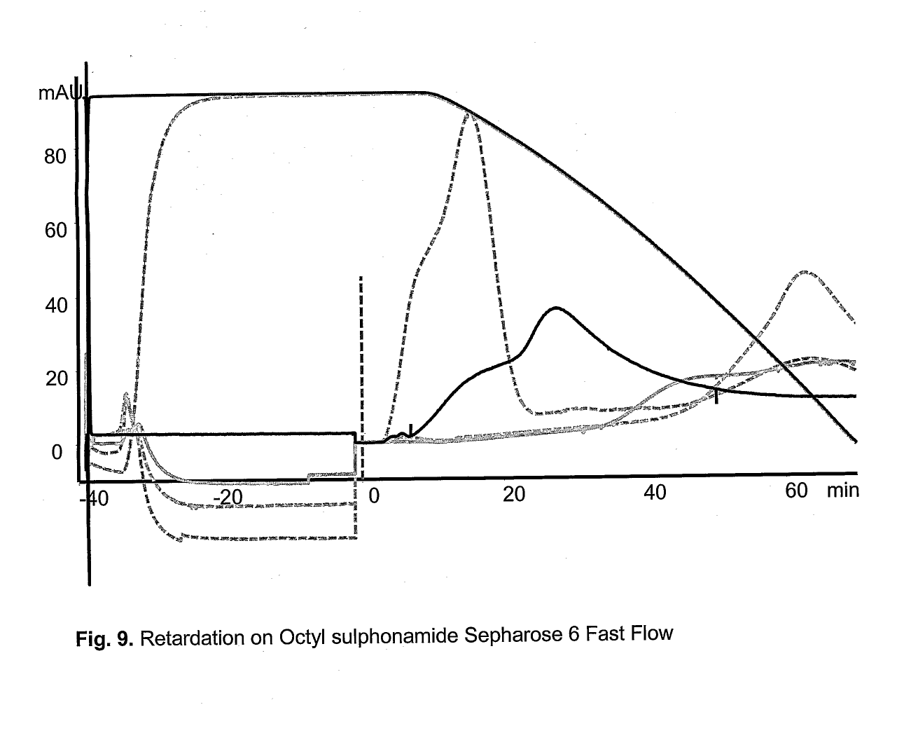

FIG. 9 shows the retardation of α-chymotrypsinogen, lysozyme, ribonuclease A and cytochrome C on Octyl sulphonamide SEPHAROSE™ 6 Fast Flow.

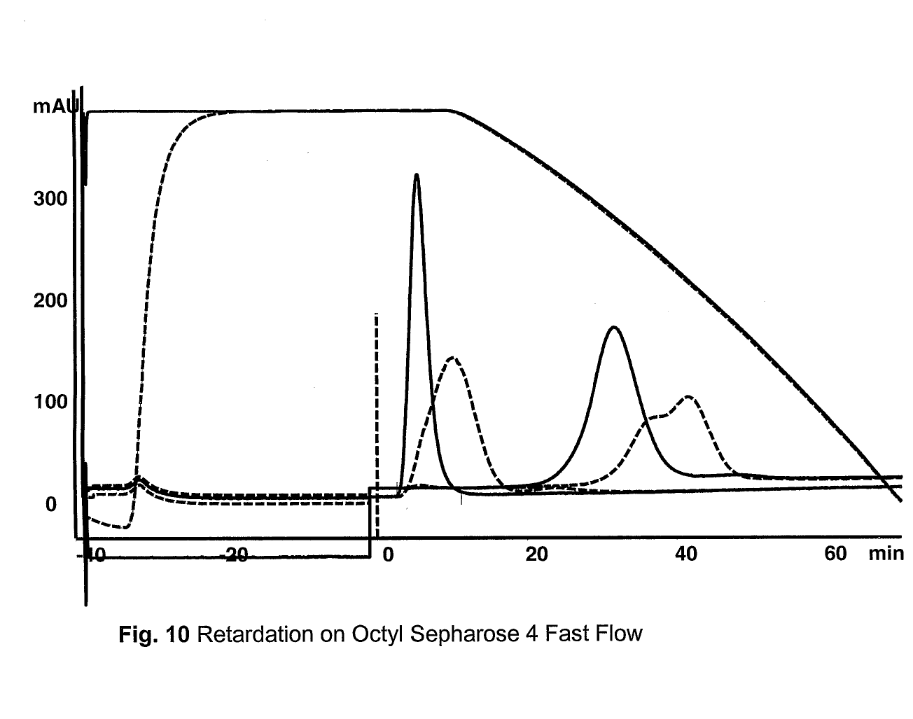

FIG. 10 shows the retardation of α-chymotrypsinogen, lysozyme, ribonuclease A and cytochrome C on Octyl SEPHAROSE™ 4 Fast Flow

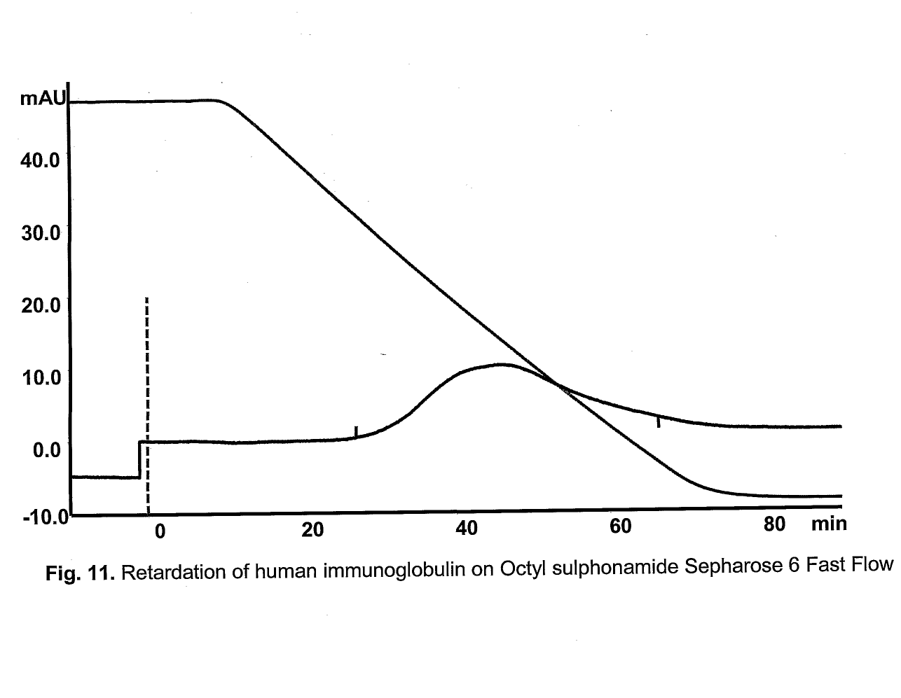

FIG. 11 shows the retardation of human immunoglobulin on Octyl sulphonamide SEPHAROSE™ 6 Fast Flow. 50 mM phosphate buffer (pH 7.0) with 0.80 M (NH₄)₂SO4 was used as buffer A and 20 mM phosphate buffer (pH 7.0)+10% isopropanol as buffer B.

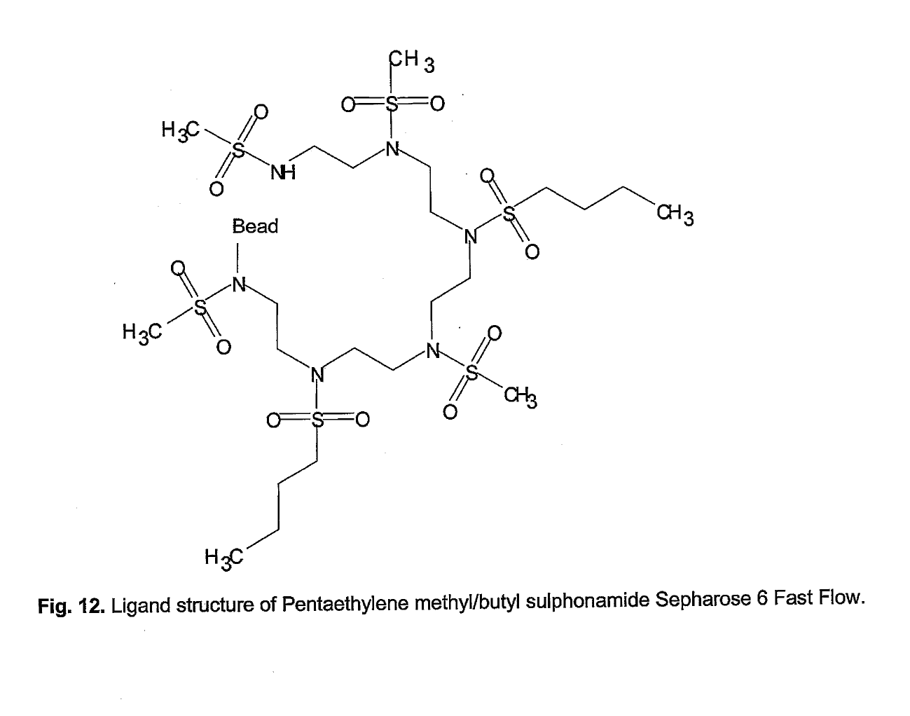

FIG. 12 shows the ligand structure of the prototype Pentaethylene methyl/butyl sulphonamide SEPHAROSE™ 6 Fast Flow.

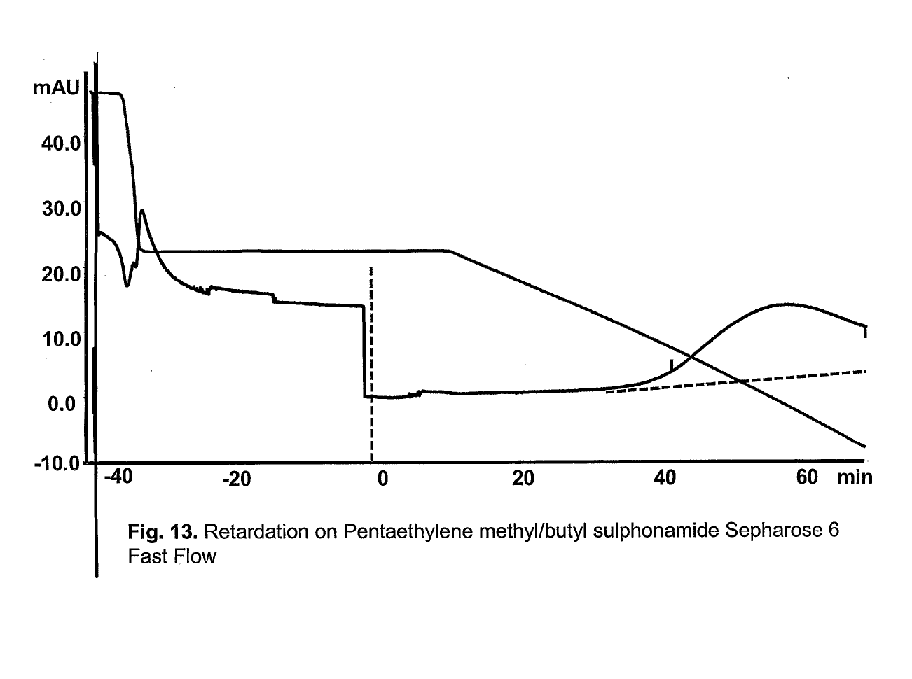

FIG. 13 shows the chromatographic retardation of human immunoglobulin on Pentaethylene methyl/butyl (80/20) sulphonamide SEPHAROSE™ 6 Fast Flow. 50 mM phosphate buffer (pH 7.0) with 0.80 M (NH₄)₂SO₄ was used as buffer A and 20 mM phosphate buffer (pH 7.0) as buffer B (see the experimental part below).

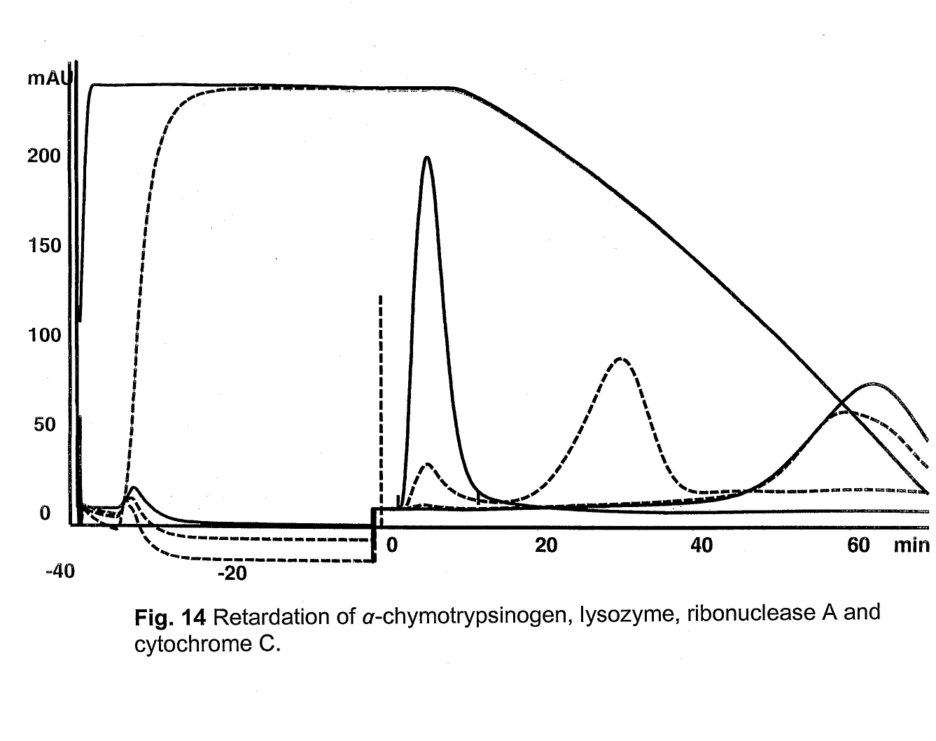

FIG. 14 shows the retardation of α-chymotrypsinogen, lysozyme, ribonuclease A and cytochrome C on Pentaethylene methyl/butyl (80/20) sulphonamide SEPHAROSE™ 6 Fast Flow.

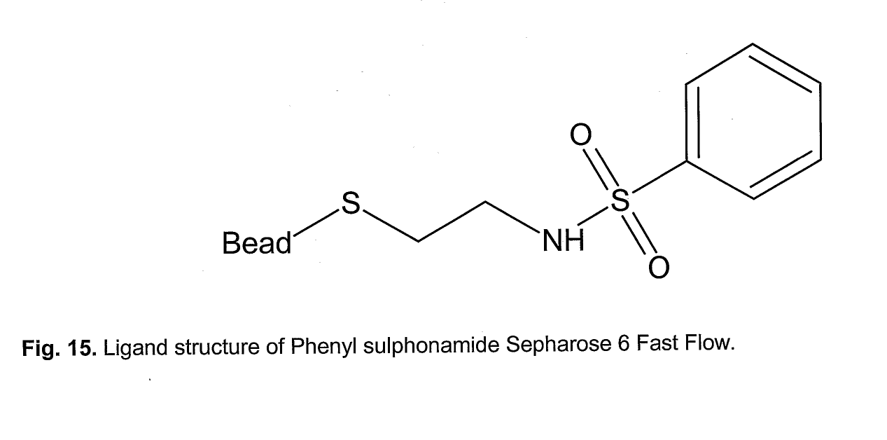

FIG. 15 shows the ligand structure of Phenyl sulphonamide SEPHAROSE™ 6 Fast Flow.

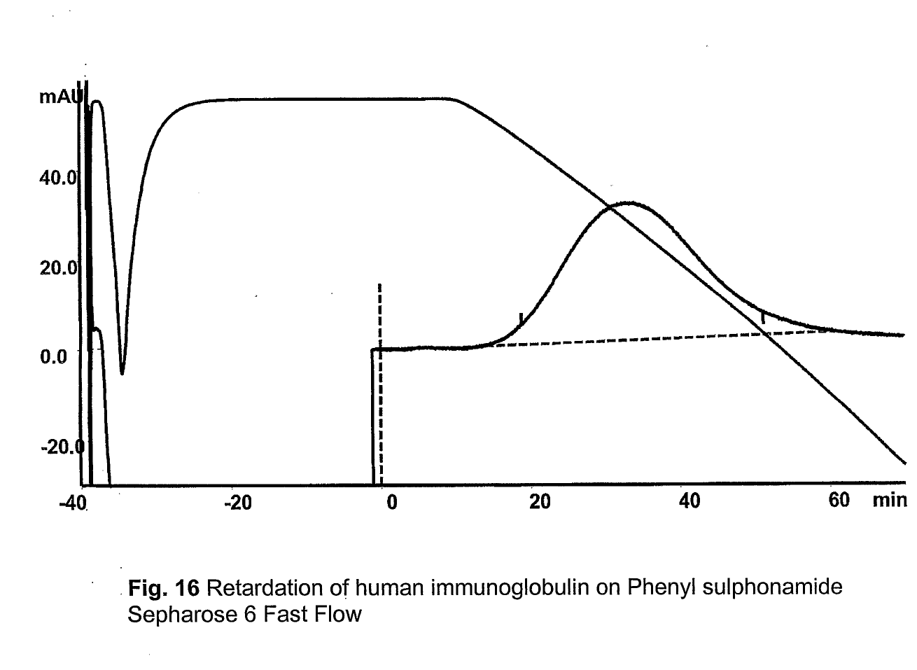

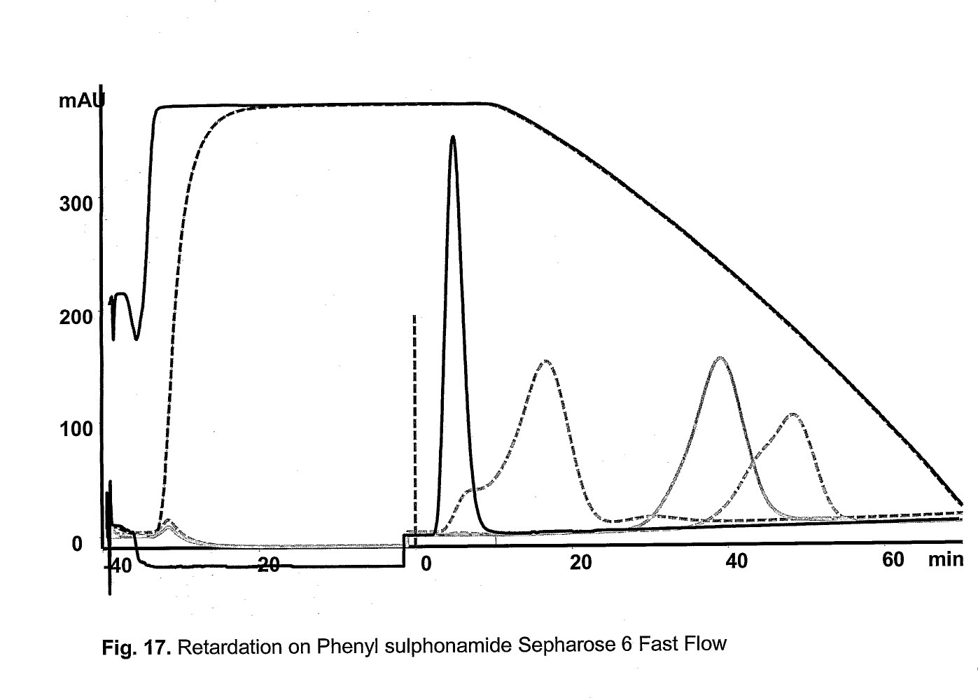

FIG. 16 shows the retardation of human immunoglobulin on Phenyl sulphonamide SEPHAROSE™ 6 Fast Flow. 50 mM phosphate buffer (pH 7.0) with 0.80 M (NH₄)₂SO4 was used as buffer A and 20 mM phosphate buffer (pH 7.0) as buffer B (see the experimental part below). FIG. 17 shows the retardation of α-chymotrypsinogen, lysozyme, ribonuclease A and cytochrome C on Phenyl sulphonamide SEPHAROSE™ 6 Fast Flow.

### EXPERIMENTAL PART

The present examples are provided for illustrative purposes only, and should not be construed as limiting the scope of the present invention as defined by the appended claims. All references given below and elsewhere in the present specification are hereby included herein by reference.

### Example 1

### Preparation of Sulphonamide HIC-Ligands

### A. Preparation of Octyl Sulphonamide SEPHAROSE™ 6 Fast Flow and Phenyl Sulphonamide SEPHAROSE™ 6 Fast Flow

Allyl Activation with Allylglycidylether

160 ml of drained SEPHAROSE™ 6 Fast Flow (GE Healthcare, Uppsala, Sweden) were transferred to a reaction vessel and 50 ml of water and 12 ml of 50% NaOH added. After 1 h of stirring at 50° C., 21 ml of allylglycidylether (AGE) were added. The reaction slurry was stirred at 50° C. for 18 h, followed by washing on a glass filter funnel with distilled water, ethanol and finally distilled water. Titration gave a degree of substitution of 21 μmol of allyl/ml of gel.

Coupling with Cysteamine

160 ml of drained gel, SEPHAROSE™ 6 Fast Flow-allyl-21 prepared as described above, were thoroughly washed with 2-propanol. The gel was stirred in a total volume of 240 ml of 2-propanol with 3.5 g of Cysteamine-HCl at 70° C. After 20 minutes, 210 mg of 2,2′Azobis(2-methylbutyronitrile) were added and after 4 hours a second 210 mg portion of 2,2′Azobis(2-methylbutyronitrile) was added to the reaction slurry.

After a total of 20 hours reaction time, the gel was washed on a glass filter with 2-propanol followed by ethanol and water. The amine content was determined by titration, 18 μmol/ml gel.

Derivatization with Octanesulfonyl Chloride

A 6 g quantity of cysteamine coupled gel (18 μmol/ml) as described above were washed with 3×10 ml ethanol followed by 3×10 ml DCM (dichloromethane). The gel was transferred to a vial with 3 ml DCM plus 5.5 equivalents of DIPEA (105 μl) and stirred for 5 minutes. After drop wise addition of 5 equivalents of Octanesulfonyl chloride (105 μl), the reaction mixture was stirred at room temperature for 18 h.

After filtration of the reaction mixture the gel was successively washed with 3×10 ml DCM, 3×10 ml ethanol and finally with 3×10 ml of distilled water.

Derivatization with Benzenesulfonyl Chloride

A 6 g quantity of the above cysteamine-coupled gel (18 μmol/ml) was washed with 3×10 ml ethanol followed by 3×10 ml DCM (dichloromethane). The gel was transferred to a vial plus 3 ml DCM with 5.5 equivalents of DIPEA (105 μl) and stirred for 5 minutes. After drop wise addition of 5 equivalents of Benzenesulfonyl chloride (70 μl), the reaction mixture was stirred at room temperature for 18 h. After filtration of the reaction mixture the gel was successively washed with 3×10 ml DCM, 3×10 ml ethanol and finally with 3×10 ml of distilled water.

### B. Preparation of Butyl Sulphonamide SEPHAROSE™ 6 Fast Flow

Allyl Activation with Allylglycidylether

110 ml of drained SEPHAROSE™ 6 Fast Flow (GE Healthcare, Uppsala, Sweden) were transferred to a reaction vessel and 40 ml of water and 4 ml of 50% NaOH were added. After 1 h of stirring at 50° C., 25 ml of allylglycidylether (AGE) were added. The reaction slurry was stirred at 50° C. for 18 h, followed by washing on a glass filter funnel with distilled water, ethanol and finally distilled water.

Titration gave a degree of substitution of 41 μmol of allyl/ml of gel.

Coupling with Cysteamine

105 ml of drained gel, Seph6FF-allyl-41 prepared as described above, was thoroughly washed with 2-propanol. The gel was stirred in a total volume of 155 ml of 2-propanol with 4.8 g of Cysteamine-HCl at 70° C. After 20 minutes, 340 mg of 2,2′Azobis(2-methylbutyronitrile) was added and after 4 hours a second 340 mg portion of 2,2′Azobis(2-methylbutyronitrile) was added to the reaction slurry.

After a total of 20 hours reaction time, the gel was washed on a glass filter with 2-propanol followed by ethanol and water. The amine content was determined by titration, 38 μmol/ml gel.

Derivatization with Butanesulfonyl Chloride

A 6 g quantity of the above cysteamine-coupled gel (38 μmol/ml) was washed with 3×10 ml ethanol followed by 3×10 ml DCM (dichloromethane). The gel was transferred to a vial plus 3 ml DCM with 5.5 equivalents of DIPEA (105 μl) and stirred for 5 minutes. After drop wise addition of 5 equivalents of Butane sulfonylchloride, the reaction mixture was stirred at room temperature for 18 h.

After filtration of the reaction mixture the gel was successively washed with 3×10 ml DCM, 3×10 ml ethanol and finally with 3×10 ml of distilled water.

C. Preparation of Pentaethylene Methyl/Butyl sulphonamide SEPHAROSE™ 6 Fast Flow
Allyl Activation with Allylglycidylether

150 ml of drained SEPHAROSE™ 6 Fast Flow (GE Healthcare, Uppsala Sweden) were suction dried on a glass filter to 106 g, and transferred to a reaction vessel and 175 ml of 50% NaOH and 20 g of Na₂SO₄ were added. After 1 h of stirring at 50° C., 175 ml of allylglycidylether (AGE) was added. The reaction slurry was stirred at 50° C. for 18 h, followed by washing on a glass filter funnel with distilled water, ethanol and finally distilled water.

Titration gave a degree of substitution of 422 μmol of allyl/ml of gel.

Amine Activation with Pentaethylenhexamine

60 ml drained allyl activated SEPHAROSE™ 6FF, 422 μmol/ml, was suspended with 50 ml distilled water in a round bottomed flask equipped with a mechanic stirrer. Sodium acetate, 5 g was added to the slurry and the mixture was stirred at room temperature for 5 min. To this bromine was added until a permanent yellow colour was obtained and then stirred for a further 5 minutes. The excess of bromine was destructed with Na-formiate. The gel was then filtered on a glass filter and washed thoroughly with water. The allyl-brominated gel was transferred into a reaction vessel, and 60 ml of pentaethylenhexamine were added, followed by pH adjustment to 12 with 50% NaOH. The reaction slurry was stirred at 50° C. for 18 h, followed by washing with water. The amine content was determined by titration, 605 μmol/ml.

Derivatization with Methane/Butane Sulfonyl Chloride

A 6 g quantity of polyamine coupled gel (605 μmol/ml) was washed with 3×10 ml ethanol followed by 3×10 ml DCM (dichloromethane). The gel was transferred to a vial plus 2 ml DCM with 3.2 equivalents of DIPEA (2.0 ml) and stirred for 5 minutes. After drop wise addition of a mixture of 675 μl Methane-sulfonylchloride and 280 μl Butanesulfonylchloride in 2 ml of DCM, the reaction mixture was stirred at room temperature for 18 h.

After filtration of the reaction mixture the gel was successively washed with 3×10 ml DCM, 3×10 ml ethanol and finally with 3×10 ml of distilled water. The derivatization procedure described above was repeated once.

### Example 2

### Separation of IgG by Hydrophobic Interaction Chromatography (HIC) Based on Sulphonamide Ligands

### A. Introduction

To test if the sulphonamide ligands adsorb human immunoglobuline (IgG) selectively, the adsorptivity of IgG and four different proteins has been tested at different conditions. The principle of the test method is that proteins are injected (50 μl) into an HR5/5 column (containing the sulphonamide ligands immobilized on SEPHAROSE™ Fast Flow) equilibrated with the A-buffer (containing a salt and a buffer component). 9.5 ml of A-buffer is then pumped through the column; then a 17.5-ml linear gradient from A-buffer to B-buffer (see below) is applied. The chromatographic profiles are then monitored at 280, 254 and 215 nm and the retention time evaluated.

### B. Experimental

Two combinations of adsorption (Buffer A#) and desorption buffers (Buffer B#) were used:
1. Buffer A1: 20 mM TRIS buffer (pH 7.5) with 1.70 M (NH₄)₂SO4
   Buffer B1: 20 mM TRIS buffer (pH 7.5)
2. Buffer A2: 50 mM phosphate buffer (pH 7.0) with 0.80 M (NH₄)₂SO4
   Buffer B2: 20 mM phosphate buffer (pH 7.0)

### C. Sample

The samples used were α-chymotrypsinogen, cytochrome C, ribonuclease A, lysozyme and human immunoglobuline (IgG, Gammanorm). The proteins were dissolved in the A-buffers. The sample concentration was 3 mg/ml for α-chymotrypsinogen, cytochrome C, lysozyme and human immunoglobuline and 9 mg/ml for ribonuclease A. Only one protein at a time was applied into the column.

### D. Instrumental

### Apparatus

| | |
|---|---|
| LC System: | ÄKTA ™ explorer 10 XT (GE Healthcare, Uppsala, |
| | Sweden) |
| Software: | UNICORN ™ (GE Healthcare, Uppsala, Sweden) |
| Injection loop: | Superloop 50 μl |
| Column: | HR 5/5 |

### Instrument Parameters

| | |
|---|---|
| Flow rate: | 0.25 ml/min |
| Detector cell: | 10 mm |
| Wavelength: | 280, 254 and 215 nm |

### E. UNICORN™ Method

### Main Method:

The column was equilibrated with the A-buffer for 38 minutes at a flow rate of 0.25 ml/min before the sample was injected. After the sample injection (50 μl) a linear gradient from 100% A-buffer to 100% B-buffer was applied. The gradient time was 70 minutes and the flow rate was 0.25 ml/min.

### F. Results and Discussion

To document if sulphonamide ligands selectively adsorb immunoglobulines, human IgG has been applied to a 1 ml column (HR 5/5) packed with these new sulphonamide adsorbents. In addition, the proteins α-chymotrypsinogen, cytochrome C, lysozyme and ribonuclease A were also applied. Two different adsorption buffers with different content of salt ((NH₄)₂SO₄) were used as adsorption buffers. Buffer A1 (20 mM TRIS buffer pH 7.5 with 1.70 M (NH₄)₂SO₄) were used for the proteins α-chymotrypsinogen, cytochrome C, lysozyme and ribonuclease A and buffer A2 (50 mM phosphate buffer pH 7.0 with 0.80 M (NH₄)₂SO₄) were used for human IgG. As B-buffer 20 mM TRIS buffer pH 7.5 or 50 mM phosphate buffer pH 7.0, respectively, was used and the gradient time was 70 minutes. The new sulphonamide ligands were attached to SEPHAROSE™ 6 Fast Flow (GE Healthcare, Uppsala, Sweden) and the chromatographic behaviour was compared to prototypes Butyl SEPHAROSE™ 4 Fast Flow and Octyl SEPHAROSE™ 4 Fast Flow.

### Chromatographic Comparison Between the Prototype Butyl Sulphonamide SEPHAROSE™ 6 Fast Flow and the Commercially Available Butyl SEPHAROSE™ 4 Fast Flow

The ligand structures of Butyl sulphonamide SEPHAROSE™ 6 Fast Flow and Butyl SEPHAROSE™ 4 Fast Flow (GE Healthcare, Uppsala, Sweden) are presented in FIG. 1. The ligand density of both media is in this experiment about 40 μmol/ml. According to FIGS. 2 and 3, IgG is interacting more strongly (longer retention time) with Butyl sulphonamide SEPHAROSE™ 6 Fast Flow (retention time: 60 min) compared to Butyl SEPHAROSE™ 4 Fast Flow (retention time: 26 min). Furthermore, the peak area observed from Butyl sulphonamide SEPHAROSE™ 6 Fast Flow is about 40% compared to the peak are obtained from Butyl SEPHAROSE™ 4 Fast Flow. The sample IgG contains subclasses of different immunoglobulins (59% of IgG 1, 36% of IgG 2, 4.9% of IgG 3 and 0.5% of IgG 4). This may indicate that for the ligands based on Butyl sulphonamide, only IgG 2 is eluted with the used gradient. The chromatograms of the proteins cytochrome C, ribonuclease A, lysozyme and α-chymotrypsinogen (FIGS. 4 and 5) show that Butyl sulphonamide SEPHAROSE™ 6 Fast Flow retards the proteins more strongly compared to Butyl SEPHAROSE™ 4 Fast Flow. Furthermore, more peaks are observed for Butyl sulphonamide SEPHAROSE™ 6 Fast Flow (FIGS. 4 and 5) indicating a better selectivity for the proteins cytochrome C, ribonuclease A, lysozyme and α-chymotrypsinogen. It can be concluded that Butyl sulphonamide SEPHAROSE™ 6 Fast Flow shows a better resolution between IgG and the other investigated proteins compared to Butyl SEPHAROSE™ 4 Fast Flow.

### Chromatographic Comparison Between the Prototype Octyl Sulphonamide SEPHAROSE™ 6 Fast Flow and the Commercially Available Octyl SEPHAROSE™ 4 Fast Flow

The ligand structures of Octyl sulphonamide SEPHAROSE™ 6 Fast Flow and Octyl SEPHAROSE™ 4 Fast Flow are presented in FIG. 6. The ligand density of Octyl sulphonamide SEPHAROSE™ 6 Fast Flow is about 20 μmol/ml and the ligand density of Octyl SEPHAROSE™ 4 Fast Flow media is 5 μmol/ml. The ligand density of the prototype Octyl sulphonamide SEPHAROSE™ 6 Fast Flow was adjusted to get retentions of the proteins cytochrome C, ribonuclease A, lysozyme and α-chymotrypsinogen comparable with Octyl SEPHAROSE™ 4 Fast Flow. According to FIGS. 7 and 8, IgG is not eluted from Octyl sulphonamide SEPHAROSE™ 6 Fast Flow but for Octyl SEPHAROSE™ 4 Fast Flow the elution peak of IgG is observed at 22 minutes. The proteins cytochrome C, ribonuclease A, lysozyme and α-chymotrypsinogen were eluted from both Octyl sulphonamide SEPHAROSE™ 6 Fast Flow and Octyl SEPHAROSE™ 4 Fast Flow as presented in FIGS. 9 and 10. This means that a very high selectivity for IgG is obtained for Octyl sulphonamide SEPHAROSE™ 6 Fast Flow. FIG. 9 also shows that ribonuclease A is eluted before cytochrome C, which shows that Octyl sulphonamide SEPHAROSE™ 6 Fast Flow has a different selectivity between these proteins compared to Octyl SEPHAROSE™ 4 Fast Flow. To verify that IgG can be eluted from Octyl sulphonamide SEPHAROSE™ 6 Fast Flow, 10% isopropanol was added to buffer B. According to FIG. 11 IgG was then eluted from the column.

### Chromatographic Performance of Sulphonamide Ligand Based on Pentaethylene SEPHAROSE™ 6 Fast Flow

One ligand prototype based on Pentaethylene methyl/butyl sulphonamide SEPHAROSE™ 6 Fast Flow is depicted in FIG. 12. The chromatographic results from IgG and the other proteins are shown in FIGS. 13 and 14. These results prove that this type of ligand gives an IgG-selectivity comparable with Butyl sulphonamide SEPHAROSE™ 6 Fast Flow (see above). It can also be noted that the separation of the proteins cytochrome C, ribonuclease A, lysozyme and α-chymotrypsinogen (FIG. 14) has a different pattern compared to Butyl SEPHAROSE™ 4 Fast Flow (FIG. 5).

### Chromatographic Performance of Phenyl Sulphonamide SEPHAROSE™ 6 Fast Flow

One ligand prototype based on Phenyl sulphonamide SEPHAROSE™ 6 Fast Flow is depicted in FIG. 15 and the chromatographic results from IgG and the other proteins are shown in FIGS. 16 and 17. The results show that α-chymotrypsinogen elute at 48.56 minutes (corresponds to a salt concentration of 0.52 M (NH₄)₂SO₄) and IgG elutes at 32.80 minutes (corresponds to a salt concentration of 0.43 M (NH₄)₂SO₄). This means that all proteins investigated (cytochrome C, ribonuclease A, lysozyme and α-chymotrypsinogen) elute at a higher ionic strength than the elution of IgG.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1. A method of isolating at least one target compound from a liquid, which method comprises the steps of:
(a) providing a mobile phase, which comprises at least one target compound and wherein the conductivity corresponds to ≧0.6 M (NH₄)₂SO₄;
(b) contacting said mobile phase with a separation matrix comprising one or more sulphonamide groups to adsorb one or more target compounds;
(c) contacting an eluent with said matrix to release one or more target compounds, wherein the conductivity of the eluent is reduced as compared to the mobile phase conductivity; and the pH is substantially equivalent to the mobile phase pH; and, optionally,
(d) recovering at least one target compound.

2. The method of claim 1, wherein step (c) is a decreasing salt gradient elution.

3. A method of isolating at least one target compound from a liquid, which method comprises the steps of:
(a) providing a mobile phase, which comprises at least one target compound and wherein the conductivity corresponds to ≧0.6 M (NH₄)₂SO₄;
(b) contacting said mobile phase with a separation matrix comprising one or more sulphonamide groups to adsorb one or more target compounds;
(c) contacting an eluent with said matrix to release one or more target compounds, wherein the eluent comprises at least one organic solvent; and, optionally,
(d) recovering at least one target compound.

4. The method of claim 3, wherein one organic solvent is isopropanol.

5. The method of claim 3, wherein the eluent comprises a decreasing salt gradient in addition to the organic solvent(s).

6. The method of claim 1 or 3, wherein the pH of the mobile phase is in the range of 6-8.

7. The method of claim 1 or 3, wherein the mobile phase comprises TRIS buffer.

8. The method of claim 1 or 3, wherein the conductivity of the mobile phase corresponds to ≧1.5, such as ≧2.0 M (NH₄)₂SO₄.

9. The method of claim 1 or 3, wherein the mobile phase comprises an ammonium salt.

10. The method of claim 1 or 3, wherein the separation matrix is comprised of a support to which one or more aliphatic sulphonamide groups have been immobilised as ligands, optionally via spacer arms.

11. The method of claim 10, wherein the aliphatic sulphonamide groups comprises 1-4 carbon atoms.

12. The method of claim 1 or 3, wherein the separation matrix is comprised of a support to which one or more aromatic sulphonamide groups have been immobilised as ligands, optionally via spacer arms.

13. The method of claim 12, wherein the ligands are substantially devoid of protonatable groups.

14. The method of claim 1 or 3, wherein the nitrogen of the sulphonamide groups of the separation matrix is derived from a primary or secondary amine.

15. The method of claim 1 or 3, wherein the sulphonamide groups of the separation matrix are monoamines.

16. The method of claim 1 or 3, wherein the sulphonamide groups of the separation matrix are polyamines.

17. The method of claim 1 or 3, wherein some or all of the sulphonamide groups are coupled to the support via nitrogen.

18. The method of claim 1 or 3, wherein some or all of the sulphonamide groups are coupled to the support via sulphur.

19. The method of claim 1 or 3, wherein the sulphonamide groups of the separation matrix are present as units of a polymer immobilised to the support.

20. The method of claim 1 or 3, wherein the support of the separation matrix is a cross-linked polysaccharide.

21. The method of claim 1 or 3, wherein the separation matrix is contained in a chromatography column.

22. The method of claim 1 or 3, wherein at least one target compound is an antibody.

23. (canceled)

24. The method of claim 22, wherein the antibody is immunoglobulin G (IgG).

25. (canceled)

26. The method of determining the quantity of at least one target compound, which method encompass a method as defined in claim 1 or 3, and in addition a subsequent step of determining the amount of target compound spectrophotometrically.

27. A kit for isolating at least one target compound from a liquid, which kit comprises, in separate compartments, a separation matrix comprising sulphonamide ligands; an TRIS buffer having a salt concentration of ≧0.6 M; and an eluent, wherein the eluent presents substantially the same pH value as the adsorption buffer and no salt.

28. The kit of claim 27, which comprises, in separate compartments, two or more different separation matrices.

29-31. (canceled)
